(19) United States
(12) Patent Application Publication
(10) Pub. No.: US 2008/0121227 A1
(43) Pub. Date: May 29, 2008

(54) Liquefaction and Saccharification Process

(75) Inventors: Swapnil Bhargava, Raleigh, NC (US); Malcolm Johal, Raleigh, NC (US); Henrik Frisner, Vaerloese (DK)

Correspondence Address: NOVOZYMES NORTH AMERICA, INC., 500 FIFTH AVENUE, SUITE 1600, NEW YORK, NY, 10110, US

(73) Assignees: Novozymes A/S, Bagsvaerd (DK); Novozymes North America, Inc., Franklinton, NC (US)

(21) Appl. No.: 11/574,357

(22) PCT Filed: Aug. 30, 2005
(86) PCT No.: PCT/US05/31101

§ 371 Date: Feb. 27, 2007

Related U.S. Application Data
(60) Provisional application No. 60/606,227, filed on Sep. 1, 2004.

(51) Int. Cl. C13J 1/06; C08B 30/00; C12P 19/20

(52) U.S. Cl. 127/46.1; 127/69; 435/96

(57) ABSTRACT

The present invention relates to a process for liquefying starch-containing material, comprising treating the starch-containing material with a bacterial alpha-amylase at a temperature between 70-90° C. for 10-120 minutes and a pullulanase at a temperature in the range from 40-60° C. for between 20 and 90 minutes. The invention also relates to a saccharification process for saccharifying liquefied starch-containing material, comprising saccharifying a liquefied starch-containing material in the presence of a carbohydrate-source generating enzyme and a pullulanase. Finally, the invention also relates to a process of producing a fermentation product, such as ethanol, comprising a liquefaction step and/or saccharification step carried out in accordance with the present invention.

### FIELD OF THE INVENTION

The present invention relates to processes of liquefying and/or saccharifying starch-containing material. The invention also relates to processes for producing fermentation products or syrups comprising liquefying and/or saccharifying starch-containing starting material in accordance with the present invention.

### BACKGROUND OF THE INVENTION

Liquefaction and saccharification are well known process steps in the art of producing fermentation products, such as ethanol, and syrups, such as glucose, high fructose syrup (HFS) and maltose, from starch-containing material.

Generally liquefaction involves gelatinization of starch simultaneously with or followed by addition of alpha-amylase in order to degrade starch into dextrins. When producing a fermentation product or syrup the liquefied starch-containing material is saccharified. Saccharification is a step in which dextrins are converted to low molecular DP₁₋₃ sugars that, e.g., can be converted or refined into syrups or metabolized by a fermenting microorganism and converted into a desired fermentation product.

EP 605,040 discloses a pullulanase derived from Bacillus deramificans for, e.g., starch saccharification with good stability over a wide temperature and pH range.

WO 00/01796 discloses a bacterial pullulanase variant which may be used for converting starch from potatoes into high fructose syrup.

Even though liquefaction and saccharification processes have been improved over the last decade there is still a need for improving such processes.

### SUMMARY OF THE INVENTION

The object of the present invention is to provide improved processes for liquefying and/or saccharifying starch-containing material suitable as steps in processes for producing syrups and fermentation products. The invention also provides fermentation product production and syrup production processes, which include liquefaction and/or saccharification steps carried out in accordance with the present invention.

The present inventors have found that when pullulanase is present during liquefaction and/or saccharification in ethanol production processes a number of advantages can be obtained. For instance, the inventors found that when liquefying milled corn under certain conditions with a bacterial alpha-amylase and a pullulanase the ethanol yield (after simultaneous saccharification and fermentation) was increased significantly compared to a corresponding process where only bacterial alpha-amylase was present. It was also found that the presence of pullulanase during saccharification also resulted in increased ethanol yields compared to a corresponding process carried out without the presence of pullulanase. This is illustrated in the Examples below.

In the first aspect the invention relates to a process of liquefying starch-containing material, comprising

(a) treating starch-containing material with a bacterial alpha-amylase at a temperature between 70-90° C. for 10-120 minutes, and

(b) treating the material obtained with a pullulanase at a temperature in the range from 40-60° C. for between 20 and 90 minutes.

In the second aspect the invention relates to a process of saccharifying liquefied starch-containing material, comprising saccharifying the liquefied starch-containing material in the presence of a carbohydrate-source generating enzyme and a pullulanase.

In the third aspect the invention relates to a process of producing a fermentation product, such as ethanol, from starch-containing material, comprising

(a) liquefying starch-containing material in accordance with the liquefaction process of the invention,

(b) saccharifying the material obtained in step (a) in the presence of a carbohydrate-source generating enzyme, and

(c) fermenting the material using a fermenting microorganism.

In the forth aspect the invention relates to a process of producing a fermentation product, such as ethanol, from starch-containing material, comprising

(a) liquefying starch-containing material with a bacterial alpha-amylase at a temperature in the range from around 70-90° C. for 15-120 minutes,

(b) saccharifying the material obtained in step (a) in accordance with a saccharification process of the invention, and

(c) fermenting the material using a fermenting microorganism.

According to the invention the process of producing a fermentation product, such as ethanol, from starch-containing material may also be carried out by

(a) liquefying starch-containing material in accordance with a liquefaction process of the invention, followed by

(b) saccharifying the material obtained in step (a) in accordance with a saccharification process of the invention

(c) fermenting the material using a fermenting microorganism.

The saccharification and fermentation steps (b) and (c) are carried out sequentially or simultaneously. In a preferred embodiment steps (b) and (c) are carried out as a simultaneous saccharification and fermentation process (SSF process).

The liquefaction and/or saccharification process of the invention may also be used for producing syrups.

### BRIEF DESCRIPTION OF THE DRAWINGS

FIG. 1 shows the ethanol yield for different liquefaction treatments based on weight loss.

FIG. 2 shows the effect of pullulanase addition in SSF on ethanol yield.

### DESCRIPTION OF THE INVENTION

The object of the present invention is to provide improved processes for liquefying and/or saccharifying starch-containing material suitable as steps in processes for producing fermentation products and syrups. The invention also relates to a process of producing a fermentation product, such as ethanol, including liquefaction and/or saccharification processes of the invention. When the end product is ethanol it may be used as, e.g., fuel ethanol; drinking ethanol, i.e., potable neutral spirits; or industrial ethanol. When the end product is syrup it is typically glucose, maltose, but may also be other syrups, such as high fructose syrup (HFS).

### Liquefaction Process of the Invention

According to the present invention, “liquefaction” is a process step in which starch-containing material, preferably milled (whole) grain, is broken down (hydrolyzed) into maltodextrins (dextrins).

The liquefying process of the invention comprises the following steps:

(a) treating starch-containing material with a bacterial alpha-amylase at a temperature between 70-90° C. for 10-120 minutes, and

(b) treating the material obtained in step (a) with a pullulanase at a temperature in the range from 40-60° C. for between 20 and 90 minutes.

The alpha-amylase may be any alpha-amylase, preferred an alpha-amylase mentioned in the section “Alpha-Amylase” below.

In a preferred embodiment step (a) is carried out at a temperature in the range from 80-90° C., preferably around 85° C., for between 60-120 minutes, preferably for between 80-100 minutes, especially around 90 minutes. In another embodiment step (a) is carried out at a temperature in the range from 80-90° C., preferably 85° C., for between 10-60 minutes, preferably between 20-50 minutes, especially around 30 minutes.

Step (b) is preferably performed at a temperature between 45-55° C., preferably around 50° C., for between 50 and 70 minutes, especially around 60 minutes. The pH during liquefaction is preferably between about 5.0 and 6.0, preferably around 5.4. The pullulanase may be any pullulanase, especially one described in the “Pullulanase”-section below. However, preferred are bacterial pullulanase, especially pullulanases derived from a strain of the genus Bacillus, especially derived from a strain of Bacillus deramificans. In a preferred embodiment the pullulanase is used in an amount of 1-100 micro g enzyme protein per g DS, especially 10-60 micro g enzyme protein per g DS.

### Starch-Containing Material

The starch-containing material used according to the present invention may be any starch-containing material. Preferred are starch-containing materials selected from the group consisting of: tubers, roots and whole grain; and any combinations thereof. In an embodiment, the starch-containing material is obtained from cereals. The starch-containing material may, e.g., be selected from the groups consisting of corn (maize), cob, wheat, barley, cassaya, sorghum, rye, milo and potato; or any combination thereof.

When the liquefaction process of the invention is included in a fermentation product production process of the invention, especially ethanol production process, the raw starch-containing material is preferably whole grain or at least mainly whole grain. The raw material may also consist of or comprise a side-stream from starch processing, e.g., C₆ carbohydrate containing process streams that are not suited for production of syrups.

### Milling

In a preferred embodiment of the invention the starch-containing material is milled or reduced in particle size in another manner before step (a) in order to open up the structure and allowing for further processing. Two processes of milling are typically used: wet and dry milling. The term “dry milling” denotes milling of the whole grain. In dry milling whole kernel is milled and used in the remaining part of the process. Wet milling gives a good separation of germ and meal (starch granules and protein) and is with a few exceptions applied at locations where there is a parallel production of syrups. Dry milling is preferred in processes aiming at producing ethanol.

The term “grinding” is also understood as milling. In a preferred embodiment of the invention dry milling is used. Other size reducing technologies such as emulsifying technology, rotary pulsation may also be used.

### Pre-Treatment

Before initiating the liquefaction process of the invention an aqueous slurry containing from 10-40 wt-%, preferably 25-35 wt-% starch-containing material, is prepared. In one embodiment the slurry is heated to a temperature in the range between 60-95° C., preferably 80-85° C.) and incubated with and without enzyme(s), such as an alpha-amylase, for initial thinning. In one embodiment the slurry may be jet-cooked at a temperature between 90-120° C., preferably around 105° C., for 1-15 minutes, preferably for 3-10 minutes, especially around 5 minutes, prior to step (a). However, it is to be understood that the process of the invention may also be carried out without initial thinning and jet-cooking.

Thus, in a particular embodiment, the liquefaction process of the invention further comprises—prior to the primary liquefaction step, i.e., prior to step (a),—the steps of:

(i) milling or reduction in particle size of starch-containing material, such as whole grain; and

(ii) forming a slurry comprising starch-containing material and water.

The liquefaction process of the invention may be followed by a standard saccharification process well known in the art or a saccharification process of the invention. This will be described in the following section.

### Saccharification Process of the Invention

“Saccharification” is a process in which maltodextrins (such as liquefied starch-containing material) is converted to low molecular sugars, such as DP₁₋₃ sugars. Saccharification of liquefied starch-containing material is well known in the art. Standard saccharification is typically performed enzymatically using at least one carbohydrate-source generating enzyme, such as especially glucoamylase.

According to the present invention liquefied starch-containing material is saccharified in the presence of carbohydrate-source generating enzyme(s) and a pullulanase. As for standard saccharification processes, a saccharification process of the invention may last up to from 20 to 100 hours, preferably about 24 to about 72 hours, and may preferably be carried out at a temperature in the range from about 30 to 65° C. and at a pH between 4 and 6, normally around pH 4.5-5.5.

It may according to the invention be preferred to do a pre-saccharification step, lasting for about 40 to 90 minutes, at a temperature in the range from 30-65° C., typically about 60° C., followed by complete saccharification during fermentation in a simultaneous saccharification and fermentation process (SSF). The most widely used process in ethanol production is the simultaneous saccharification and fermentation (SSF) process, in which there is no holding stage for saccharification, meaning that the fermenting organism, such as yeast, and enzyme(s) is(are) added together. When the process is carried out as a simultaneous saccharification and fermentation process (SSF process) the temperature used in typically in the range from 30-40° C., preferably around 32° C.

In a preferred embodiment the carbohydrate-source generating enzyme is a glucoamylase, preferably one derived from a strain of the genus Aspergillus, preferably A. niger, A. awamori or A. oryzae, or a strain of Talaromyces, preferably a strain of Talaromyes emersonii or a strain of Athelia, preferably Athelia rolfsii (previously denoted Corticium rolfsii—see, e.g., U.S. Pat. No. 4,727,026). A glucoamylase may suitably be added in amounts of between 0.005-2 AGU/g DS, preferably 0.01-1 AGU/g DS, such as especially around 0.3 AGU/g DS.

The pullulanase may be any pullulanase, preferably a bacterial pullulanase, preferably derived from a strain of the genus Bacillus, especially derived from a strain of Bacillus deramificans. In a preferred embodiment the pullulanase is used in an amount between 1-100 micro g enzyme protein per g DS, preferably between 10-60 micro g enzyme protein per g DS.

### Fermentation Product Production Process

A fermentation product production process of the invention generally involves the steps of liquefaction, saccharification, fermentation and optionally recovering the product, preferably by distillation.

According to this aspect, the invention relates to a process of producing a fermentation product from starch-containing material, comprising

(a) liquefying starch-containing material in accordance with a liquefaction process of the invention,

(b) saccharifying the material obtained in step (a) in the presence of a carbohydrate-source generating enzyme, and

(c) fermenting the material using a fermenting microorganism.

In an embodiment the saccharification and fermentation steps are carried out sequentially or simultaneously. In a preferred embodiment steps (b) and (c) are carried out as a simultaneous saccharification and fermentation process (SSF process). In a preferred embodiment of the invention starch-containing raw material, such as whole grain, preferably corn, is dry milled in order to open up the structure and allow for further processing.

In a preferred embodiment step (a) is carried out at a temperature in the range from 80-90° C., preferably around 85° C., for between 60-120 minutes, preferably for between 80 and 100 minutes, especially around 90 minutes. In a preferred embodiment the pH during liquefaction is between about 5.0 and 6.0, preferably around 5.4. The bacterial alpha-amylase may be any of the alpha-amylases mentioned in the “Alpha-Amylase”-section above.

In another aspect, of the invention relates to a process of producing a fermentation product from starch-containing material, comprising

(a) liquefying starch-containing material with a bacterial alpha-amylase at a temperature in the range from around 70-90° C. for 15-120 minutes,

(b) saccharifying the material obtained in step (a) in accordance with a saccharification process of the invention, and

(c) fermenting the material using a fermenting microorganism.

In an embodiment the saccharification and fermentation steps are carried out sequentially or simultaneously. In a preferred embodiment the saccharification and fermentation is carried out as a SSF process.

In a further aspect a fermentation product production process of the invention includes both a liquefaction and a saccharification process of the invention. Thus, in this aspect the fermentation product is produced by

(a) liquefying starch-containing material in accordance with a liquefaction process of the invention,

(b) saccharifying the material obtained in step (a) in accordance with a saccharification process of the invention, and

(c) fermenting the material using a fermenting microorganism.

In an embodiment the saccharification and fermentation steps are carried out sequentially or simultaneously. In a preferred embodiment the saccharification and fermentation are carried out as a SSF process.

### Fermentation

The term “fermenting microorganism” refers to any organism suitable for use in a desired fermentation process. Suitable fermenting microorganisms are according to the invention capable of fermenting, i.e., converting, preferably DP₁₋₃ sugars, such as especially glucose and maltose, directly or indirectly into ethanol. Examples of fermenting microorganisms include fungal organisms, such as yeast. Preferred yeast includes strains of the Saccharomyces spp., and in particular Saccharomyces cerevisiae. Commercially available yeast includes, e.g., RED STAR®/Lesaffre Ethanol Red (available from Red Star/Lesaffre, USA), SUPERSTART (available from Alltech), GERT STRAND (available from Gert Strand AB, Sweden) and FERMIOL (available from DSM Specialties). In an embodiment, yeast is applied to the saccharified mash. However, is it preferred that the saccharification and fermentation is carried out simultaneously. Fermentation is ongoing for 24-96 hours, such as typically 35-65 hours. In a preferred embodiment, the temperature is between 26-34° C., in particular about 32° C., and the pH is generally from pH 3-6, preferably around pH 4-5. Yeast cells are preferably applied in amounts of 10⁵ to 10¹², preferably from 10⁷ to 10¹⁰, especially 5×10⁷ viable yeast count per mL of fermentation broth. During ethanol producing phase the yeast cell count should preferably be in the range from 10⁷ to 10¹⁰, especially around 2×10⁸. Further guidance in respect of using yeast for fermentation can be found in, e.g., “The alcohol Textbook” (Editors K. Jacques, T. P. Lyons and D. R. Kelsall, Nottingham University Press, United Kingdom 1999), which is hereby incorporated by reference.

### Recovery of the Fermentation Product

Optionally the fermentation product, such as ethanol, is recovery after fermentation, preferably by including the step of

(d) distillation to obtain the fermentation product; wherein the fermentation in step (c) and the distillation in step (d) is carried out simultaneously or separately/sequential; optionally followed by one or more process steps for further refinement of the fermentation product, such as ethanol.

### Alpha-Amylase

According to the invention preferred alpha-amylases are of bacterial or fungal origin. In a preferred embodiment the Bacillus alpha-amylase is derived from a strain of B. licheniformis, B. amyloliquefaciens, B. subtilis or B. stearothermophilus, but may also be derived from other Bacillus sp., such as a strain of the Bacillus sp. NCIB 12289, NCIB 12512, NCIB 12513 or DSM 9375, all of which are described in detail in WO 95/26397, and the alpha-amylase described by Tsukamoto et al., Biochemical and Biophysical Research Communications, 151 (1988), pp. 25-31.

Specific examples of contemplated alpha-amylases include the Bacillus licheniformis alpha-amylase shown in SEQ ID NO: 4, the Bacillus amyloliquefaciens alpha-amylase SEQ ID NO: 5 and the Bacillus stearothermophilus alpha-amylase shown in SEQ ID NO: 3 in WO 99/19467 (all sequences hereby incorporated by reference). In an embodiment of the invention the alpha-amylase may be an enzyme having a degree of identity of at least 60%, preferably 70%, more preferred 80%, even more preferred 90%, such as at least 95%, at least 96%, at least 97%, at least 98% or at least 99% to any of the sequences shown in SEQ ID NO: 1, 2 or 3 in WO 99/19467.

The Bacillus alpha-amylase may also be a variant and/or hybrid, especially one described in any of WO 96/23873, WO 96/23874, WO 97/41213, WO 99/19467, WO 00/60059, and WO 02/10355 (all documents hereby incorporated by reference). Specifically contemplated alpha-amylase variants are disclosed in U.S. Pat. Nos. 6,093,562, 6,297,038 and 6,187,576 (hereby incorporated by reference) and include Bacillus stearothermophilus alpha-amylase (BSG alpha-amylase) variants having a deletion of one or two amino acid in positions R179 to G182, preferably a double deletion disclosed in WO 1996/023873-see e.g., page 20, lines 1-10 (hereby incorporated by reference), preferably corresponding to delta(181-182) compared to the wild-type BSG alpha-amylase amino acid sequence set forth in SEQ ID NO:3 disclosed in WO 99/19467 or deletion of amino acids R179 and G180 using SEQ ID NO:3 in WO 99/19467 for numbering (which reference is hereby incorporated by reference). Even more preferred are Bacillus alpha-amylases, especially Bacillus stearothermophilus alpha-amylase, which have a double deletion corresponding to delta(181-182) and further comprise a N193F substitution (also denoted I181*+G182*+N193F) compared to the wild-type BSG alpha-amylase amino acid sequence set forth in SEQ ID NO:3 disclosed in WO 99/19467 (hereby incorporated by reference).

A hybrid alpha-amylase specifically contemplated comprises 445 C-terminal amino acid residues of the Bacillus licheniformis alpha-amylase (shown in SEQ ID NO: 4 of WO 99/19467) and the 37 N-terminal amino acid residues of the alpha-amylase derived from Bacillus amyloliquefaciens (shown in SEQ ID NO: 5 of WO 99/19467), with one or more, especially all, of the following substitutions: G48A+T491+G107A+H156Y+A181 T+N190F+I201 F+A209V+Q264S (using Bacillus licheniformis numbering in SEQ ID NO: 4 of WO 99/19467). Also preferred are variants having one or more of the following mutations (or corresponding mutations in other Bacillus alpha-amylase backbones): H154Y, A181T, N190F, A209V and Q264S and/or deletion of two residues between positions 176 and 179, preferably deletion of E178 and G179 (using the SEQ ID NO: 5 numbering of WO 99/19467).

The bacterial alpha-amylase may be added in amounts as are well-known in the art. When measured in KNU units the alpha-amylase activity is preferably present in an amount of 0.0005-5 KNU per g DS, preferably 0.001-1 KNU per g DS, such as around 0.050 KNU per g DS.

Fungal alpha-amylases include alpha-amylases derived from a strain of Aspergillus, such as Aspergillus oryzae, Aspergillus niger, and A. kawashii alpha-amylases. In a preferred embodiment, the alpha-amylase is an acid alpha-amylase. In a more preferred embodiment the acid alpha-amylase is an acid fungal alpha-amylase or an acid bacterial alpha-amylase. More preferably, the acid alpha-amylase is an acid fungal alpha-amylase derived from the genus Aspergillus. A commercially available acid fungal amylase is SP288 (available from Novozymes A/S, Denmark).

In an embodiment the alpha-amylase is an acid alpha-amylase. The term “acid alpha-amylase” means an alpha-amylase (E.C. 3.2.1.1) which added in an effective amount has activity at a pH in the range of 3.0 to 7.0, preferably from 3.5 to 6.0, or more preferably from 4.0-5.0.

A preferred acid fungal alpha-amylase is a Fungamyl-like alpha-amylase. In the present disclosure, the term “Fungamyl-like alpha-amylase” indicates an alpha-amylase which exhibits a high identity, i.e., more than 50%, 55%, 60%, 65%, 70%, 75%, 80%, 85% 90%, 95 or even 99% identical to the amino acid sequence shown in SEQ ID NO: 10 in WO 96/23874 (hereby incorporated y reference).

Preferably the alpha-amylase is an acid alpha-amylase, preferably from the genus Aspergillus, preferably of the species Aspergillus niger. In a preferred embodiment the acid fungal alpha-amylase is the one from A. niger disclosed as “AMYA_ASPNG” in the Swiss-prot/TeEMBL database under the primary accession no. P56271 (Hereby incorporated by reference). Also variants of said acid fungal amylase having at least 70% identity, such as at least 80% or even at least 90%, 95%, 96%, 97%, 98% or 99% identity thereto is contemplated.

A fungal acid alpha-amylase is preferably added in an amount of 0.001-10 AFAU/g of DS, in an amount of 0.01-0.25 AFAU/g of DS, or more preferably in an amount of 0.05-0.20 AFAU/kg of DS, such as around 0.1 AFAU/k DS.

Preferred commercial alpha-amylases include MYCOLASE™ from DSM; BAN™, TERMAMYL™ SC, FUNGAMYL™, LIQUOZYME™ X and SAN™ SUPER, SAN™ EXTRA L from Novozymes A/S, Denmark) and CLARASE™ L-40,000, DEX-LO™, SPEYME FRED, SPEZYME™ AA, and SPEZYME™ DELTA AA (Genencor Int., USA), and the acid fungal alpha-amylase sold under the trade name SP 288 (available from Novozymes A/S, Denmark).

### Carbohydrate-Source Generating Enzyme

The term “carbohydrate-source generating enzyme” includes glucoamylase (being glucose generators), beta-amylase and maltogenic amylase (being maltose generators). A carbohydrate-source generating enzyme is capable of producing a carbohydrate that can be used as an energy-source by the fermenting microorganism(s) in question, for instance, when used in a process of the invention for producing a fermentation product, such as ethanol. The generated carbohydrate may be converted directly or indirectly to the desired fermentation product, preferably ethanol. According to the invention a mixture of carbohydrate-source generating enzymes may be used. Especially contemplated mixtures are mixtures of at least a glucoamylase and an alpha-amylase, especially an acid amylase, even more preferred an acid fungal alpha-amylase. The ratio between acidic fungal alpha-amylase activity (AFAU) per glucoamylase activity (AGU) (AFAU per AGU) may in an embodiment of the invention be at least 0.1, in particular at least 0.16, such as in the range from 0.12 to 0.50 or more.

### Glucoamylase

A glucoamylase used according to the invention may be derived from any suitable source, e.g., derived from a microorganism or a plant. Preferred glucoamylases are of fungal or bacterial origin, e.g., selected from the group consisting of Aspergillus glucoamylases, in particular A. niger G1 or G2 glucoamylase (Boel et al. (1984), EMBO J. 3 (5), p. 1097-1102), or variants thereof, such as one disclosed in WO 92/00381, WO 00/04136, WO 01/04273 and WO 03/029449 (from Novozymes, Denmark, hereby incorporated by reference); the A. awamori glucoamylase (WO 84/02921), A. oryzae (Agric. Biol. Chem. (1991), 55 (4), p. 941-949), or variants or fragments thereof.

Other Aspergillus glucoamylase variants include variants to enhance the thermal stability: G137A and G139A (Chen et al. (1996), Prot. Eng. 9, 499-505); D257E and D293E/Q (Chen et al. (1995), Prot. Engng. 8, 575-582); N182 (Chen et al. (1994), Biochem. J. 301, 275-281); disulphide bonds, A246C (Fierobe et al. (1996), Biochemistry, 35, 8698-8704; and introduction of Pro residues in position A435 and S436 (Li et al. (1997), Protein Engng. 10, 1199-1204. Other glucoamylases include Athelia rolfsii (previously denoted Corticium rolfsii) glucoamylase (see U.S. Pat. No. 4,727,026 and (Nagasaka, Y. et al. (1998) Purification and properties of the raw-starch-degrading glucoamylases from Corticium rolfsii, Appl Microbiol Biotechnol 50:323-330), Talaromyces glucoamylases, in particular, derived from Talaromyces emersonii (WO 99/28448), Talaromyces leycettanus (U.S. Pat. No. Re. 32,153), Talaromyces duponti, Talaromyces thermophilus (U.S. Pat. No. 4,587,215). Bacterial glucoamylases contemplated include glucoamylases from the genus Clostridium, in particular C. thermoamylolyticum (EP 135,138), and C. thermohydrosulfuricum (WO 86/01831).

Commercially available compositions comprising glucoamylase include AMG 200L; AMG 300 L; SAN™ SUPER, SAN™ EXTRA L, SPIRIZYME™ PLUS, SPIRIZYME™ FUEL, SPIRIZYME™ B4U and AMG™ E (from Novozymes A/S); OPTIDEX™ 300 (from Genencor Int.); AMIGASE™ and AMIGASE™ PLUS (from DSM); G-ZYME™ G900, G-ZYME™ and G990 ZR (from Genencor Int.).

Glucoamylase may in an embodiment be added in an amount of 0.005-2 AGU/g DS, preferably between 0.01-1 AGU/g DS, such as especially around 0.3 AGU/g DS.

### Beta-Amylase

At least according to the invention the a beta-amylase (E.C 3.2.1.2) is the name traditionally given to exo-acting maltogenic amylases, which catalyze the hydrolysis of 1,4-alpha-glucosidic linkages in amylose, amylopectin and related glucose polymers. Maltose units are successively removed from the non-reducing chain ends in a step-wise manner until the molecule is degraded or, in the case of amylopectin, until a branch point is reached. The maltose released has the beta anomeric configuration, hence the name beta-amylase.

Beta-amylases have been isolated from various plants and microorganisms (W. M. Fogarty and C. T. Kelly, Progress in Industrial Microbiology, vol. 15, pp. 112-115, 1979). These beta-amylases are characterized by having optimum temperatures in the range from 40° C. to 65° C. and optimum pH in the range from 4.5 to 7. A commercially available beta-amylase from barley is NOVOZYM™ WBA from Novozymes A/S, Denmark and SPEZYME™ BBA 1500 from Genencor Int., USA.

### Maltogenic Amylase

The amylase may also be a maltogenic alpha-amylase. A “maltogenic alpha-amylase” (glucan 1,4-alpha-maltohydrolase, E.C. 3.2.1.133) is able to hydrolyze amylose and amylopectin to maltose in the alpha-configuration. A maltogenic amylase from Bacillus stearothermophilus strain NCIB 11837 is commercially available from Novozymes A/S under the tradename MALTOGENASE™. Maltogenic alpha-amylases are described in U.S. Pat. Nos. 4,598,048, 4,604,355 and 6,162,628, which are hereby incorporated by reference.

The maltogenic amylase may in a preferred embodiment be added in an amount of 0.05-5 mg total protein/gram DS or 0.05-5 MANU/g DS.

### Pullulanase

Pullulanases (E.C. 3.2.1.41, pullulan 6-glucano-hydrolase), are debranching enzymes characterized by their ability to hydrolyze the alpha-1,6-glycosidic bonds in, for example, amylopectin and pullulan.

Specifically contemplated pullulanases according to the present invention include the pullulanases from Bacillus amyloderamificans disclosed in U.S. Pat. No. 4,560,651 (hereby incorporated by reference), the pullulanase disclosed as SEQ ID NO: 2 in WO 01/151620 (hereby incorporated by reference), the Bacillus deramificans disclosed as SEQ ID NO: 4 in WO 01/151620 (hereby incorporated by reference), and the pullulanase from Bacillus acidopullulyticus disclosed as SEQ ID NO: 6 in WO 01/151620 (hereby incorporated by reference) and also described in FEMS Mic. Let. (1994) 115, 97-106.

The pullulanase may according to the invention be added in an effective amount which include the preferred range of from between 1-100 micro g per g DS, especially from 10-60 micro g per g DS. Pullulanase activity may be determined as NPUN. An Assay for determination of NPUN is described in the “Materials & Methods”-section below.

Suitable commercially available pullulanase products include PROMOZYME D, PROMOZYME™ D2 (Novozymes A/S, Denmark), OPTIMAX L-300 (Genencor Int., USA), and AMANO 8 (Amano, Japan).

### Starch Conversion

The liquefaction and/or saccharification processes of the invention may also be included in a starch conversion process for producing syrups such as glucose, maltose, fructose, malto-oligosaccharides and isomalto-oligosaccharides.

Therefore, in one aspect, the invention relates to a process of producing a syrup from starch-containing material comprising

(a) liquefying starch-containing material with a bacterial alpha-amylase at a temperature in the range from around 70-90° C. for 15-120 minutes, and

(b) saccharifying the material obtained in step (a) using a carbohydrate-source generating enzyme.

In a preferred embodiment step (a) is carried out at a temperature in the range from 80-90° C., preferably around 85° C., for between 60-120 minutes, preferably for between 80 and 100 minutes, especially around 90 minutes. In a preferred embodiment the pH during liquefaction is between about 5.0 and 6.0, preferably around 5.4. The bacterial alpha-amylase may be any of the alpha-amylases mentioned in the “Alpha-Amylase”-section above.

In another aspect the invention relates to a process of producing syrup from starch-containing material comprising

(a) liquefying starch-containing material using an alpha-amylase, and

(b) saccharifying the material obtained in step (a) in accordance with a saccharification process of the invention.

The saccharified material may then be refined, further converted and/or recovered into syrup using one or more steps well-know in the art. In the case of high fructose syrup the saccharified material may further be isomerized using an isomerase enzyme.

The syrup product may be a syrup including glucose, maltose, fructose, malto-oligosaccharides and isomalto-oligosaccharides.

### Production of Enzymes

The enzymes referenced herein may be derived or obtained from any suitable origin, including, bacterial, fungal, yeast or mammalian origin. The term “derived” or means in this context that the enzyme may have been isolated from an organism where it is present natively, i.e., the identity of the amino acid sequence of the enzyme are identical to a native enzyme. The term “derived” also means that the enzymes may have been produced recombinantly in a host organism, the recombinant produced enzyme having either an identity identical to a native enzyme or having a modified amino acid sequence, e.g., having one or more amino acids which are deleted, inserted and/or substituted, i.e., a recombinantly produced enzyme which is a mutant and/or a fragment of a native amino acid sequence or an enzyme produced by nucleic acid shuffling processes known in the art. Within the meaning of a native enzyme are included natural variants. Furthermore, the term “derived” includes enzymes produced synthetically by, e.g., peptide synthesis. The term “derived” also encompasses enzymes which have been modified e.g., by glycosylation, phosphorylation, or by other chemical modification, whether in vivo or in vitro. The term “obtained” in this context means that the enzyme has an amino acid sequence identical to a native enzyme. The term encompasses an enzyme that has been isolated from an organism where it is present natively, or one in which it has been expressed recombinantly in the same type of organism or another, or enzymes produced synthetically by, e.g., peptide synthesis. With respect to recombinantly produced enzymes the terms “obtained” and “derived” refers to the identity of the enzyme and not the identity of the host organism in which it is produced recombinantly.

The enzymes may also be purified. The term “purified” as used herein covers enzymes free from other components from the organism from which it is derived. The term “purified” also covers enzymes free from components from the native organism from which it is obtained. The enzymes may be purified, with only minor amounts of other proteins being present. The expression “other proteins” relate in particular to other enzymes. The term “purified” as used herein also refers to removal of other components, particularly other proteins and most particularly other enzymes present in the cell of origin of the enzyme of the invention. The enzyme may be “substantially pure,” that is, free from other components from the organism in which it is produced, that is, for example, a host organism for recombinantly produced enzymes. In preferred embodiment, the enzymes are at least 75% (w/w) pure, more preferably at least 80%, at least 85%, at least 90%, at least 95%, at least 96%, at least 97%, at least 98%, or at least 99% pure. In another preferred embodiment, the enzyme is 100% pure.

The enzymes used according to the present invention may be in any form suitable for use in the processes described herein, such as, e.g., in the form of a dry powder or granulate, a non-dusting granulate, a liquid, a stabilized liquid, or a protected enzyme. Granulates may be produced, e.g., as disclosed in U.S. Pat. Nos. 4,106,991 and 4,661,452, and may optionally be coated by process known in the art. Liquid enzyme preparations may, for instance, be stabilized by adding stabilizers such as a sugar, a sugar alcohol or another polyol, lactic acid or another organic acid according to established process. Protected enzymes may be prepared according to the process disclosed in EP 238,216.

Even if not specifically mentioned in context of a method or process of the invention, it is to be understood that the enzyme(s) or agent(s) is(are) used in an “effective amount”.

### Materials and Methods

### Enzymes:

Bacterial Alpha-Amylase A: Bacillus stearothermophilus alpha-amylase variant with the mutations: 1181*+G182*+N193F disclosed in U.S. Pat. No. 6,187,576 and available on request from Novozymes A/S, Denmark.

### Glucoamylase TN: Glucoamylase derived from Talaromyces emersonii and disclosed as SEQ ID NO: 7 in WO 99/28448 with side activity of Aspergillus niger glucoamylase and Aspergillus niger acid alpha-amylase.

### Pullulanase A: Bacillus deramificans pullulanase available as PROMOZYME™ D2 from Novozymes A/S, Denmark.

### Yeast

### Red Star™ available from Red Star/Lesaffre, USA

### Methods:

### Alpha-Amylase Activity (KNU)

The amylolytic activity may be determined using potato starch as substrate. This method is based on the break-down of modified potato starch by the enzyme, and the reaction is followed by mixing samples of the starch/enzyme solution with an iodine solution. Initially, a blackish-blue color is formed, but during the break-down of the starch the blue color gets weaker and gradually turns into a reddish-brown, which is compared to a colored glass standard.

One Kilo Novo alpha amylase Unit (KNU) is defined as the amount of enzyme which, under standard conditions (i.e., at 37° C. +/−0.05; 0.0003 M Ca²⁺; and pH 5.6) dextrinizes 5260 mg starch dry substance Merck Amylum solubile.

A folder EB-SM-0009.02/01 describing this analytical method in more detail is available upon request to Novozymes A/S, Denmark, which folder is hereby included by reference.

### Determination of Pullulanase Activity (NPUN)

Endo-pullulanase activity in NPUN is measured relative to a Novozymes pullulanase standard. One pullulanase unit (NPUN) is defined as the amount of enzyme that releases 1 micro mol glucose per minute under the standard conditions (0.7% red pullulan (Megazyme), pH 5, 40° C., 20 minutes). The activity is measured in NPUN/ml using red pullulan.

1 ml diluted sample or standard is incubated at 40° C. for 2 minutes. 0.5 ml 2% red pullulan, 0.5 M KCl, 50 mM citric acid, pH 5 are added and mixed. The tubes are incubated at 40° C. for 20 minutes and stopped by adding 2.5 ml 80% ethanol. The tubes are left standing at room temperature for 10-60 minutes followed by centrifugation 10 minutes at 4000 rpm. OD of the supernatants is then measured at 510 nm and the activity calculated using a standard curve.

### Determination of FAU Activity

One Fungal Alpha-Amylase Unit (FAU) is defined as the amount of enzyme, which breaks down 5.26 g starch (Merck Amylum solubile Erg. B.6, Batch 9947275) per hour based upon the following standard conditions:

| | |
|---|---|
| Substrate | Soluble starch |
| Temperature | 37° C. |
| pH | 4.7 |
| Reaction time | 7-20 minutes |

### Determination of Acid Alpha-Amylase Activity (AFAU)

Acid alpha-amylase activity is measured in AFAU (Acid Fungal Alpha-amylase Units), which are determined relative to an enzyme standard.

The standard used is AMG 300 L (from Novozymes A/S, Denmark, glucoamylase wild-type Aspergillus niger G1, also disclosed in Boel et al. (1984), EMBO J. 3 (5), p. 1097-1102) and WO 92/00381). The neutral alpha-amylase in this AMG falls after storage at room temperature for 3 weeks from approx. 1 FAU/mL to below 0.05 FAU/mL.

The acid alpha-amylase activity in this AMG standard is determined in accordance with the following description. In this method, 1 AFAU is defined as the amount of enzyme, which degrades 5.260 mg starch dry matter per hour under standard conditions.

Iodine forms a blue complex with starch but not with its degradation products. The intensity of color is therefore directly proportional to the concentration of starch. Amylase activity is determined using reverse colorimetry as a reduction in the concentration of starch under specified analytic conditions.

Alpha-amylase

| | | |
|---|---|---|
| Starch + Iodine | → | Dextrins + Oligosaccharides |
| | 40° C., pH 2.5 | |
| Blue/violet | t = 23 sec. | Decoloration |

Standard conditions/reaction conditions: (per minute)

| | |
|---|---|
| Substrate: | Starch, approx. 0.17 g/L |
| Buffer: | Citate, approx. 0.03 M |
| Iodine (I₂): | 0.03 g/L |
| CaCl₂: | 1.85 mM |
| pH: | 2.50 ± 0.05 |
| Incubation temperature: | 40° C. |
| Reaction time: | 23 seconds |
| Wavelength: | lambda = 590 nm |
| Enzyme concentration: | 0.025 AFAU/mL |
| Enzyme working range: | 0.01-0.04 AFAU/mL |

If further details are preferred these can be found in EB-SM-0259.02/01 available on request from Novozymes A/S, Denmark, and incorporated by reference.

### Glucoamylase Activity (AGU)

The Novo Glucoamylase Unit (AGU) is defined as the amount of enzyme, which hydrolyzes 1 micromole maltose per minute under the standard conditions 37° C., pH 4.3, substrate: maltose 23.2 mM, buffer: acetate 0.1 M, reaction time 5 minutes.

An autoanalyzer system may be used. Mutarotase is added to the glucose dehydrogenase reagent so that any alpha-D-glucose present is turned into beta-D-glucose. Glucose dehydrogenase reacts specifically with beta-D-glucose in the reaction mentioned above, forming NADH which is determined using a photometer at 340 nm as a measure of the original glucose concentration.

| AMG incubation: | |
|---|---|
| Substrate: | maltose 23.2 mM |
| Buffer: | acetate 0.1 M |
| pH: | 4.30 ± 0.05 |
| Incubation temperature: | 37° C. ± 1 |
| Reaction time: | 5 minutes |
| Enzyme working range: | 0.5-4.0 AGU/mL |

| Color reaction: | |
|---|---|
| GlucDH: | 430 U/L |
| Mutarotase: | 9 U/L |
| NAD: | 0.21 mM |
| Buffer: | phosphate 0.12 M; 0.15 M NaCl |
| pH: | 7.60 ± 0.05 |
| Incubation temperature: | 37° C. ± 1 |
| Reaction time: | 5 minutes |
| Wavelength: | 340 nm |

A folder (EB-SM-0131.02/01) describing this analytical method in more detail is available on request from Novozymes A/S, Denmark, which folder is hereby included by reference.

### Determination of Maltogenic Amylase Activity (MANU)

One MANU (Maltogenic Amylase Novo Unit) may be defined as the amount of enzyme required to release one micro mole of maltose per minute at a concentration of 10 mg of maltotriose (Sigma M 8378) substrate per ml of 0.1 M citrate buffer, pH 5.0 at 37° C. for 30 minutes.

### Determination of Identity Between Two Sequences

The degree of identity between two amino acid sequences is determined by the Clustal method (Higgins, 1989, CABIOS 5: 151-153) using the LASERGENE™ MEGALIGN™ software (DNASTAR, Inc., Madison, Wis.) with an identity table and the following multiple alignment parameters: Gap penalty of 10, and gap length penalty of 10. Pairwise alignment parameters were Ktuple=1, gap penalty=3, windows=5, and diagonals=5].

### EXAMPLES

### Example 1

### The Effect of Pullulanase Addition During Liquefaction on SSF Performance

To test the effect of pullulanase addition in liquefaction three different liquefactions were carried out. Initially ground (milled) corn was used to make 30% slurry with tap water. The pH in all three liquefactions was adjusted to 5.4 using diluted H₂SO₄. In the first liquefaction (control), Bacterial Alpha-Amylase A (50 NU/g DS) was added and kept at 85° C. for 1.5 hours. In the second liquefaction, the same procedure was followed, except that the incubation time with Bacterial Alpha-Amylase A at 85° C. was reduced to 0.5 hours, the temperature was reduced and Pullulanase A was added (37 micro g of enzyme protein/g DS). The mixture was then kept at 50° C. for 1 hour. To make comparison the same temperature profiling was used with Bacterial Alpha-Amylase A (i.e., 85° C. and 50° C. temperature stages). Once the liquefaction was complete, the reactions were stopped by adding H₂SO₄ (40%). Samples were withdrawn to analyze the sugar profiles (using HPLC) and DE values. The liquefied samples were frozen and later subjected to SSF.

The effect of liquefaction treatment on SSF was evaluated via mini-scale fermentations. Samples after liquefaction were thawed and the pH was adjusted to 5.0 with diluted NaOH. Approximately 4 g of mash was added to 16 ml polystyrene tubes (Falcon 352025). Tubes were then dosed with Glucoamylase TN (0.5 AGU/g DS). Five replicates of each treatment were run. After dosing the tubes with enzyme, they were inoculated with 0.04 mL/g mash of yeast (Red Star™) propagate that had been grown for 21 hours on corn mash. Vials were capped with a screw on lid which had been punctured with a very small needle to allow gas release and vortexed briefly before weighing and incubation at 32° C. Fermentation progress was followed by weighing the tubes over time. Tubes were vortexed briefly before each weighing. Weight loss values were converted to ethanol yield (g ethanol/g DS) by the following formula:

$$g ethanol / g DS = \frac{\begin{matrix}g {CO}_{2} weight loss \frac{1 mol {CO}_{2}}{44.0098 g {CO}_{2}} \\ \frac{1 mol ethanol}{1 mol {CO}_{2}} \frac{46.094 g ethanol}{1 mol ethanol}\end{matrix}}{g corn in tube \% DS of corn}$$

### DE Measurement

After the liquefaction step, 0.2 g of the sample is added to 50 mL of water. The sample was further diluted to bring OD450 between 0.2 and 0.8 (the linear range of the glucose standards used). To 200 microL of the diluted sample 0.8 mL of reagent A (60 g/L Na₂CO₃, 16 g/L Glycine, 450 mg/L CUSO₄.5H₂O) and 0.8 mL reagent B (1.2 g/L Neocuproine) were added in duplicate. Samples were boiled for 12 minutes then stopped by placing in ice cold water for 5 minutes. Finally 3.2 mL of water is added to bring the volume up to 5 mL. After mixing the samples, they are measured at OD450 nm. DE is calculated based on the following formula, where DS is measured from the original liquefaction material:

DE+micro g glucose/mL/(g of original sample in 100 mL*DS in the sample)

### HPLC Analysis

Approximately 1 mL of cleared supernatant was passed through a 0.45 micro M filter to remove solids. A 1/10 dilution of this sample was analyzed by HPLC for glucose, maltose, maltotriose and larger soluble sugars (DP₄₊) and ethanol.

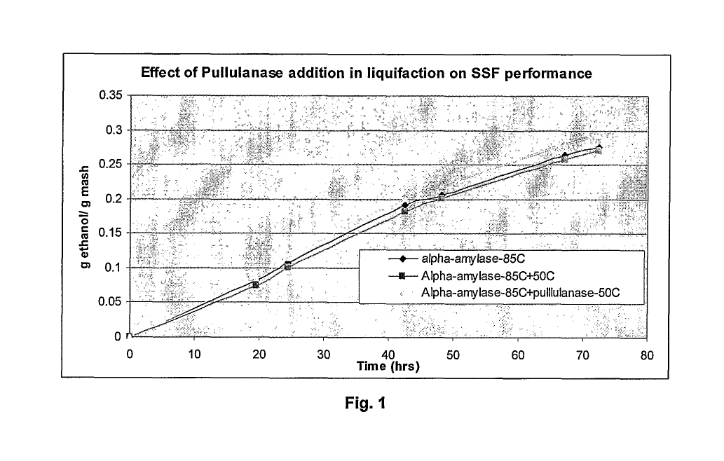

FIG. 1 and Table 1 show that the presence of pullulanase during liquefaction results in a significant increase in ethanol yields after 72 hours and has a positive impact on ethanol production during fermentation. Further, pullulanase addition increases the DE from 8.2 to 12.5 and smaller sugars were released.

TABLE 1

| Effect of secondary | | | | |
|---|---|---|---|---|
| enzymes on Alpha- | g | % | Range of | |
| Amylase A liquefaction in | Ethanol/g | increase in | % increase | |
| ethanol yield | DS | Ethanol yield | over control | |
| Bacterial Alpha-Amylase A | 0.2715 | 0.00% | −2.15% | 2.19% |
| 85° C.-50° C. (control) | | | | |
| Bacterial Alpha-Amylase A | 0.2930 | 7.94% | 5.27% | 10.67% |
| 85° C./Pullulanase (0.20) | | | | |
| 50° C. | | | | |

### Example 2

### The Effect of Pullulanase Addition During SSF Ethanol Production

Liquefaction was carried out with Bacterial Alpha-Amylase A only. Similar to Example 1, ground (milled) corn was used to make 30% slurry with tap water. The pH in all liquefactions was adjusted to 5.4 using diluted H₂SO₄. Bacterial Alpha-Amylase A (50 NU/g DS) was added and kept at 85° C. for 1.5 hours. Once the liquefaction was complete, the reactions were stopped by adding H₂SO₄ (40%). Samples were withdrawn to analyze the sugar profiles (using HPLC) and DE values. The liquefied samples were frozen and later subjected to SSF.

The effect of liquefaction treatment on SSF was evaluated via mini-scale fermentations. Samples after liquefaction were thawed and the pH was adjusted to 5.0 with diluted NaOH. Approximately 4 g of mash was added to 16 mL polystyrene tubes (Falcon 352025). In control runs, tubes were dosed with Glucoamylase TN (0.1 AGU/g DS). Pullulanase A was added dosed based on equivalent enzyme protein of 0.1 AGU of purified glucoamylase. Six replicates of each treatment were run. After dosing the tubes with enzyme, they were inoculated with 0.04 mL/g mash of yeast (RED STAR™) propagate that had been grown for 21 hours on corn mash. Vials were capped with a screw on lid which had been punctured with a very small needle to allow gas release and vortexed briefly before weighing and incubation at 32° C. Fermentation progress was followed by weighing the tubes over time. Tubes were vortexed briefly before each weighing. Weight loss values were converted to ethanol yield (g ethanol/g DS) by the following formula:

$$g ethanol / g DS = \frac{\begin{matrix}g {CO}_{2} weight loss \frac{1 mol {CO}_{2}}{44.0098 g {CO}_{2}} \\ \frac{1 mol ethanol}{1 mol {CO}_{2}} \frac{46.094 g ethanol}{1 mol ethanol}\end{matrix}}{g corn in tube \% DS of corn}$$

### HPLC Analysis

Approximately 1 mL of cleared supernatant was passed through a 0.45 micro M filter to remove solids. A 1/10 dilution of this sample was analyzed by HPLC for glucose, maltose, maltotriose and larger soluble sugars (DP₄₊) and ethanol.

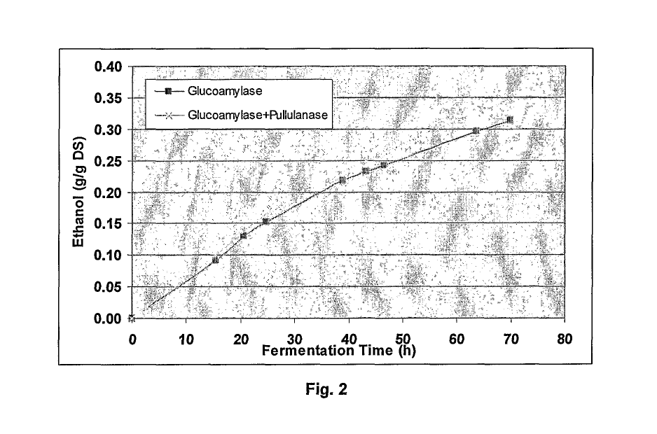

Weight loss data show (FIG. 2) that the presence of pullulanase during simultaneous saccharification and fermentation (SSF) results in a significantly improved ethanol yield.

### CONCLUSION

Addition of pullulanase in either liquefaction or simultaneous saccharification and fermentation (SSF) results in significant improved ethanol yield.

1-53. (canceled)

54. A process for liquefying starch-containing material, comprising
(a) treating starch-containing material with a bacterial alpha-amylase at a temperature between 70-90° C. for 10-120 minutes, and
(b) treating the material obtained in step (a) with a pullulanase at a temperature in the range from 40-60° C. for between 20 and 90 minutes.

55. The process of claim 54, wherein step (a) is carried out at a temperature in the range from 80-90° C.

56. The process of claim 54, wherein step (b) is performed at a temperature between 45-55° C.

57. The process of claim 54 wherein the pH during liquefaction is between about 5.0 and 6.0.

58. The process of claim 54, wherein the pullulanase is a bacterial pullulanase.

59. The process of claim 54, wherein the pullulanase is derived from a strain of Bacillus deramificans.

60. The process of claim 54, further comprising prior to step (a) the steps of
(i) milling of starch-containing material; and
(ii) forming a slurry comprising the milled material and water.

61. The process of claim 60, wherein the milling step is a dry milling step.

62. The process of claim 60, wherein the milling step is a wet milling step.

63. The process of claim 54, further comprising subjecting the liquefied material to saccharification and fermentation.

64. The process of claim 63, wherein the saccharification and fermentation are simultaneous saccharification and fermentation (SSF).

65. A process for saccharifying liquefied starch-containing material, comprising saccharifying the liquefied starch-containing material in the presence of a carbohydrate-source generating enzyme and a pullulanase.

66. The process of claim 65, wherein the carbohydrate-source generating enzyme is a glucoamylase.

67. The process of claim 65, wherein the glucoamylase is present in an amount in the range from 0.005-2 AGU/g DS of glucoamylase.

68. The process of claim 65, further comprising subjecting the saccharified material to fermentation using a fermenting microorganism.

69. The process of claim 68, wherein the saccharification and fermentation is carried out as simultaneous saccharification and fermentation (SSF).

70. The process of claim 69, wherein the simultaneous saccharification and fermentation (SSF) are carried out at a temperature between 30-40° C.

71. The process of claim 68, wherein the fermenting organism is yeast.

72. A process of producing a fermentation product from starch-containing material, comprising
(a) liquefying starch-containing material as defined in claim 54,
(b) saccharifying the material obtained in step (a) in the presence of a carbohydrate-source generating enzyme, and
(c) fermenting the material using a fermenting microorganism.

73. A process for producing a fermentation product from starch-containing material, comprising
(a) liquefying starch-containing material with a bacterial alpha-amylase at a temperature in the range from around 70-90° C. for 15-120 minutes
(b) saccharifying the material obtained in step (a) as defined in claim 65, and
(c) fermenting the material using a fermenting microorganism.
